(19) United States
(12) Patent Application Publication
(10) Pub. No.: US 2009/0297643 A1
(43) Pub. Date: Dec. 3, 2009

(54) BOTANICAL COMPOSITION AND ITS USES

(75) Inventor: Rishi Agarwal, Uttrakhand (IN)

Correspondence Address: BROOKS KUSHMAN P.C., 1000 TOWN CENTER, TWENTY-SECOND FLOOR, SOUTHFIELD, MI, 48075, US

(73) Assignee: MICRO-DOSE LIFE SCIENCES, LLC, Novi, MI (US)

(21) Appl. No.: 12/236,018

(22) Filed: Sep. 23, 2008

(30) Foreign Application Priority Data
Jun. 2, 2008 (IN) 1340/CHE/2008

(51) Int. Cl. A61K 36/886; A61K 36/00

(52) U.S. Cl. 424/744; 424/725

(57) ABSTRACT

This invention describes a novel combination of botanical components for relieving one or more symptoms related to hemorrhoids. The composition includes a therapeutically effective amount of an admixture including an extract from the bark of Negundo aceroides; and parts or extracts of Blumea odorata.

### CROSS-REFERENCE TO RELATED APPLICATIONS

This application claims foreign priority benefits under 35 U.S.C. §119(a)-(d) to 1340/CHE/2008 filed Jun. 2, 2008, which is hereby incorporated by reference in its entirety.

### BACKGROUND OF THE INVENTION

1. Field of the Invention

The present invention relates to botanical compositions that are useful for treating one or more symptoms of hemorrhoids.

2. Background Art

Hemorrhoids, or piles, refer to a condition in which the veins around the anus or lower rectum are swollen and inflamed. When a dilated vein forms a mass on the outer side of the external sphincter at the lower end of the anal canal, or is covered by the skin of the anal canal, this is referred to as an external hemorrhoid. When the swollen vein is beneath the mucous membrane at the top of the anal canal and within the sphincter, the condition is known as an internal hemorrhoid. Many factors appear to be implicated in the formation of hemorrhoids including: excessive straining during defecation, prolonged sitting or standing, pregnancy, constipation, obesity, dietary insufficiencies, disease, and as a consequence of normal aging. Whatever the cause, however, hemorrhoid sufferers experience considerable discomfort from hemorrhoid symptoms such as; pain, itching, burning and rectal bleeding.

With the onset of hemorrhoids, medical treatment is initially aimed at relieving these symptoms. For mild to moderate discomfort, over-the-counter creams, ointments or pads, or suppositories containing local anesthetics, vasoconstrictors, antiseptics, emollient protectants, topical anti-inflammatory agents (containing hydrocortisone), and stool softeners are often prescribed. In these types of treatments, the anesthetic preparations are used to relieve the pain and itching associated with hemorrhoids. Astringents, on the other hand, are used to shrink or constrict the swollen tissue, while anti-inflammatory substances reduce inflammation. Often, these creams and ointments will also contain an antiseptic to help prevent infection. A stool softener can be added to reduce strain on the affected tissue and produce a less irritating vehicle. Although some treatments on the market contain one or two of these biologically effective remedies, there is a need to combine these characteristics into one natural formulation. For more severe or persistent hemorrhoid symptoms, physical techniques are sometimes used to remove or reduce the size of hemorrhoids. These include; rubber band ligation, where a rubber band is placed around the base of the hemorrhoid inside the rectum in order to cut off the circulation and cause the hemorrhoid to wither away; sclerotherapy, is a treatment in which a chemical solution is injected around the blood vessel to shrink the hemorrhoid; infrared coagulation, involves the burning of hemorrhoidal tissue; hemorrhoidectomy, is the surgical removal of a hemorrhoid; and stapling, which blocks the flow of blood to hemorrhoidal tissue and is generally considered less painful than surgery.

Several patents and publications describe the use of plant extracts in formulations for treating or alleviating hemorrhoids. For example:

U.S. Pat. No. 4,192,866, issued Mar. 11, 1980 describes a preparation for the treatment of anorectal diseases, especially hemorrhoids, comprising polyglycerides and ripe berry products of the plant Solanum carolinese or Horse Nettle.

U.S. Pat. No. 4,761,285, issued Aug. 2, 1988 describes a composition for the relief and treatment of hemorrhoids made from Leptandra Culver's root, chick peas and grape seeds, which may be taken orally or made into a salve.

U.S. Pat. No. 4,877,781, issued Oct. 31, 1989 describes a treatment compound that includes pharmaceutically effective amounts of hydrocortisone compound, pramoxine hydrogen chloride, and ephedrine. The compound may further contain witch hazel, glycerin, vitamin A, and propylene glycol.

U.S. Pat. No. 6,228,387, issued May 8, 2001 describes a topical and oral treatment combination for the healing of hemorrhoids. The composition for topical administration includes gotu kola extract, grape seed extract, horse chestnut extract, aloe vera, vitamin C, and vitamin E. The composition for oral administration to be used in combination with the topical administration includes; gotu kola extract, grape seed extract, bilberry extract, vitamin C and vitamin E.

U.S. Pat. No. 6,565,850, issued May 20, 2003 is directed to gel compositions useful in the preparation of medicated gel products suitable for the treatment of anorectal disorders such as hemorrhoids. This includes the use of an topical administration of a gel consisting of hamamelis water up to 50% plus a carrier.

U.S. Pat. No. 6,653,352, issued Nov. 25, 2003, is directed toward a composition containing capsicium extract together with other ingredients to neutralize the discomfort from the application of capsicium extract to the skin enabling the treatment of many types of discomforts including, arthritis pain, neuropathy, post surgical scarring, hemorrhoid pain and itching, and pruritis.

Accordingly, the invention provides a composition that may be taken orally or made into a liquid, gel, cream, ointment, salve or suppository.

### SUMMARY OF THE INVENTION

The present invention describes a novel combination of botanicals for the natural treatment of blind and bleeding piles and fissures, as well as, associated conditions such as; pain, constipation, and backache. It has been discovered that the combination of botanicals described herein provides unexpected beneficial results in the treatment of one or more symptoms associated with blind and bleeding piles.

The invention provides safe and effective methods of treating and/or delaying the progression of hemorrhoids and/or their associated symptoms in a patient in need thereof by administering a therapeutically affective amount of the botanical of the invention.

### DETAILED DESCRIPTION OF THE PREFERRED EMBODIMENT(S)

Reference will now be made in detail to presently preferred compositions, embodiments and methods of the present invention, which constitute the best modes of practicing the invention presently known to the inventors. However, it is to be understood that the disclosed embodiments are merely exemplary of the invention that may be embodied in various and alternative forms. Therefore, specific details disclosed herein are not to be interpreted as limiting, but merely as a representative basis for any aspect of the invention and/or as a representative basis for teaching one skilled in the art to variously employ the present invention.

Except in the examples, or where otherwise expressly indicated, all numerical quantities in this description indicating amounts of material or conditions of reaction and/or use are to be understood as modified by the word “about” in describing the broadest scope of the invention.

It is also to be understood that this invention is not limited to the specific embodiments and methods described below, as specific components and/or conditions may, of course, vary. Furthermore, the terminology used herein is used only for the purpose of describing particular embodiments of the present invention and is not intended to be limiting in any way.

It must also be noted that, as used in the specification and the appended claims, the singular form “a,” “an,” and “the” comprise plural referents unless the context clearly indicates otherwise. For example, reference to a component in the singular is intended to comprise a plurality of components.

Throughout this application, where publications are referenced, the disclosures of these publications in their entireties are hereby incorporated by reference into this application to more fully describe the state of the art to which this invention pertains.

As used herein “pharmaceutically acceptable” means that the designated carrier, vehicle, diluent, is excipient(s), is generally chemically and/or physically compatible with the other ingredients comprising the botanical composition, and physiologically compatible with the recipient thereof.

As used herein, “tincture” refers to an alcoholic extract or alcohol/water extract of a material, in particular, a herb, plant or tree component. The tincture used herein is typically the mother tincture of a component prepared according to the procedures of the Homœopathic Pharmacopœia of the United States (“HPUS”). Procedures for forming the mother tincture are also found in German Homoeopathic Pharmacopoeia (“GHP”), 5 edition (Nov. 3, 2003), Medpharm, ISBN-10: 3887630955, ISBN-13: 978-3887630959. This procedure from (“GHP”) is hereby incorporated by reference. When prepared according these procedures, a component is subject to the extracting solvent for an extended period of time. This results in tinctures with reproducible amounts of ingredients.

As used herein, “Trituration” refers to a process of combining the botanical composition with sugar of milk.

Ingredients of the botanical composition may be described by the Decimal Scale or the Centesimal Scale. The Decimal Scale is based on the principle that first potency (i.e., dilution) should contain 1/10th part of the original drug and each succeeding potency should contain 1/10th of the previous potency. In a variation, this scale is based on the mother tincture. The decimal potency is denoted by a suffix including the letter ‘X’ to the number indicating the potency. Therefore, the first potency is 1X, the second potency is 2X and so on. The Centesimal Scale is based on the principle that first potency (i.e., dilution) should contain 1/100th part of the original drug and each succeeding potency should contain 1/100th of the previous potency. The Centesimal scale is denoted by simply affixing the numerical after the name of the drug like Hamamelis 200, Millifolium 30, Nux. Vom. 200, etc. In a variation, this scale is also based on the mother tincture. These two systems for potency are also described in German Homoeopathic Pharmacopoeia (“GHP”), 5 edition (Nov. 3, 2003), Medpharm, ISBN-10: 3887630955, ISBN-13: 978-3887630959. These descriptions from (“GHP”) are hereby incorporated by reference.

In one embodiment of the present invention, a botanical composition for treating a symptom of hemorrhoids is provided. The botanical composition of this embodiment includes a therapeutically effective amount of a part of or an extract from Acer negundo L. Acer negundo L is also known as Box-elder or Ash-leaved maple. In particular, the extract used in the present embodiment is from the bark of this tree. In a refinement, the extract is provided as a tincture, and in particular, a tincture in 65% alcohol/water (v/v). (v/v means volume/volume). Prior to forming the botanical composition of the invention, the tincture is optionally diluted with a suitable solvent (e.g., alcohol, water/alcohol). In one refinement, material from Acer negundo L is present in an amount greater than or equal to, in increasing order of preference, 0.5 weight %, 0.005 weight %, 0.01 weight %, 0.03 weight %, 0.04 weight %, of the total weight of the botanical composition. In another refinement, material from Acer negundo L is present in an amount less than or equal to, in increasing order of preference, 20 weight %, 10 weight %, 8 weight %, 5 weight %, and 2 weight % of the total weight of the botanical composition. The homeopathic composition Negundum 2X may be used as the source for Acer negundo L. Therefore, in a variation of the present embodiment, the botanic composition includes Negundum 2X in an amount from about 0.1 to about 10 weight % of the total weight botanical composition. In a refinement of this variation, the amount of material from Acer negundo L in the botanical composition are equal to the amounts in this range of Negundum 2X no matter the source. In another variation of the present embodiment, the botanic composition includes Negundum 2X in an amount from about 1 to about 8 weight % of the total weight botanical composition. In a refinement of this variation, the amount of material from Acer negundo L in the botanical composition is equal to the amounts in this range of Negundum 2X no matter the source. In still another variation of the present embodiment, the botanic composition includes Negundum 2X in an amount from about 2 to about 7 weight % of the total weight botanical composition. In a refinement of this variation, the amount of material from Acer negundo L in the botanical composition is equal to the amounts in this range of Negundum 2X no matter the source.

In a variation of the present embodiment, the botanical composition further includes parts or extracts of Blumea odorata (“Blumea”). Blumea odorata is an herb commonly found in India. In a refinement, the extract is provided as a tincture, and in particular, a tincture in 65% alcohol/water (v/v). (v/v means volume/volume). Prior to forming the botanical composition of the invention, the tincture is optionally diluted with a suitable solvent (e.g., alcohol, water/alcohol). In one refinement, material from Blumea odorata is present in an amount greater than or equal to, in increasing order of preference, 0.5 weight %, 0.005 weight %, 0.01 weight %, 0.03 weight %, 0.04 weight %, of the total weight of the botanical composition. In another refinement, material from Blumea odorata is present in an amount less than or equal to, in increasing order of preference, 20 weight %, 10 weight %, 8 weight %, 5 weight %, and 2 weight % of the total weight of the botanical composition. The homeopathic composition Blumea OD 2X may be used as the source for Blumea odorata. Therefore, in a variation of the present embodiment, the botanic composition includes Blumea OD 2X in an amount from about 1 to about 15 weight % of the total weight botanical composition. In a refinement of this variation, the amount of material from Blumea odorata in the botanical composition are equal to the amounts in this range of Blumea OD 2X no matter the source. In another variation of the present embodiment, the botanic composition includes Blumea OD 2X in an amount from about 4 to about 10 weight % of the total weight botanical composition. In a refinement of this variation, the amount of material from Blumea odorata in the botanical composition is equal to the amounts in this range of Blumea OD 2X no matter the source. In still another variation of the present embodiment, the botanic composition includes Blumea OD 2X in an amount from about 7 to about 9 weight % of the total weight botanical composition. In a refinement of this variation, the amount of material from Blumea odorata in the botanical composition is equal to the amounts in this range of Blumea OD 2X no matter the source.

In another variation of the present embodiment, the botanical composition further comprises parts or extracts of Krameria triandra Ruiz (also known as Krameria triandra Pav.) This shrub is also known as Rhatany or Peruvian rhatany. Typically, an extract of the root of Krameria triandra Ruiz is used herein. In a refinement, the extract is provided as a tincture, and in particular, a tincture in 65% alcohol/water (v/v). (v/v means volume/volume). Prior to forming the botanical composition of the invention, the tincture is optionally diluted with a suitable solvent (e.g., alcohol, water/alcohol). In one refinement, material from Krameria triandra Ruiz is present in an amount greater than or equal to, in increasing order of preference, 0.5 weight %, 0.005 weight %, 0.01 weight %, 0.03 weight %, 0.04 weight %, of the total weight of the botanical composition. In another refinement, material from Krameria triandra Ruiz is present in an amount less than or equal to, in increasing order of preference, 20 weight %, 10 weight %, 8 weight %, 5 weight %, and 2 weight % of the total weight of the botanical composition. The homeopathic composition Rhatanhia 200 may be used as the source for Krameria triandra Ruiz. Therefore, in a variation of the present embodiment, the botanic composition includes Rhatanhia 200 in an amount from about 0.1 to about 10 weight % of the total weight botanical composition. In a refinement of this variation, the amount of material from Krameria triandra Ruiz in the botanical composition are equal to the amounts in this range of Rhatanhia 200 no matter the source. In another variation of the present embodiment, the botanic composition includes Rhatanhia 200 in an amount from about 2 to about 8 weight % of the total weight botanical composition. In a refinement of this variation, the amount of material from Krameria triandra Ruiz in the botanical composition is equal to the amounts in this range of Rhatanhia 200 no matter the source. In still another variation of the present embodiment, the botanic composition includes Rhatanhia 200 in an amount from about 3 to about 7 weight % of the total weight botanical composition. In a refinement of this variation, the amount of material from Krameria triandra Ruiz in the botanical composition is equal to the amounts in this range of Rhatanhia 200 no matter the source.

In another variation of the present embodiment, the botanical composition further comprises parts or extracts of Hamamelis virginica. Hamamelis virginica is also known as Witch hazel. Typically, an extract of the bark of Hamamelis virginica is used herein. In a refinement, the extract is provided as a tincture, and in particular, a tincture in 65% alcohol/water (v/v). (v/v means volume/volume). Prior to forming the botanical composition of the invention, the tincture is optionally diluted with a suitable solvent (e.g., alcohol, water/alcohol). In one refinement, Hamamelis virginica is present in an amount greater than or equal to, in increasing order of preference, 0.1 weight %, 0.0001 weight %, 0.0005 weight %, 0.0007 weight %, 0.001 weight %, of the total weight of the botanical composition. In another refinement, Hamamelis virginica is present in an amount less than or equal to, in increasing order of preference, 20 weight %, 10 weight %, 8 weight %, 5 weight %, and 2 weight % of the total weight of the botanical composition. The homeopathic composition Hamamelis 200 may be used as the source Hamamelis virginica. Therefore, in a variation of the present embodiment, the botanic composition includes Hamamelis 200 in an amount from about 1 to about 15 weight % of the total weight botanical composition. In a refinement of this variation, the amount of material from Hamamelis virginica in the botanical composition are equal to the amounts in this range of Hamamelis 200 no matter the source. In another variation of the present embodiment, the botanic composition includes Hamamelis 200 in an amount from about 4 to about 10 weight % of the total weight botanical composition. In a refinement of this variation, the amount of material from Hamamelis virginica in the botanical composition is equal to the amounts in this range of Hamamelis 200 no matter the source. In still another variation of the present embodiment, the botanic composition includes Hamamelis 200 in an amount from about 7 to about 9 weight % of the total weight botanical composition. In a refinement of this variation, the amount of material from Hamamelis virginica in the botanical composition is equal to the amounts in this range of Hamamelis 200 no matter the source.

In another variation of the present embodiment, the botanical composition further comprises parts or extracts of Ficus religiosa L. (“Ficus Rel.”). Ficus religiosa L., also known as Peepul, is a species of banyan fig native to Sri Lanka, Nepal, India, southwest China, Indochina, and Vietnam. Typically, an extract of the bark of Ficus religiosa L is used herein. In a refinement, the extract is provided as a tincture, and in particular, a tincture in 65% alcohol/water (v/v). (v/v means volume/volume). Prior to forming the botanical composition of the invention, the tincture is optionally diluted with a suitable solvent (e.g., alcohol, water/alcohol). In one refinement, Ficus religiosa L is present in an amount greater than or equal to, in increasing order of preference, 0.5 weight %, 0.005 weight %, 0.01 weight %, 0.03 weight %, 0.04 weight %, of the total weight of the botanical composition. In another refinement, Ficus religiosa L is present in an amount less than or equal to, in increasing order of preference, 20 weight %, 10 weight %, 8 weight %, 5 weight %, and 2 weight % of the total weight of the botanical composition. The homeopathic composition Ficus rel. 2X may be used as the source for Ficus religiosa L. Therefore, in a variation of the present embodiment, the botanic composition includes Ficus rel. 2X in an amount from about 1 to about 15 weight % of the total weight botanical composition. In a refinement of this variation, the amount of material from Ficus religiosa L. in the botanical composition are equal to the amounts in this range of Ficus rel. 2X no matter the source. In another variation of the present embodiment, the botanic composition includes Ficus rel. 2X in an amount from about 4 to about 10 weight % of the total weight botanical composition. In a refinement of this variation, the amount of material from Ficus religiosa L. in the botanical composition is equal to the amounts in this range of Ficus rel. 2X no matter the source. In still another variation of the present embodiment, the botanic composition includes Ficus rel. 2X in an amount from about 7 to about 9 weight % of the total weight botanical composition. In a refinement of this variation, the amount of material from Ficus religiosa L. in the botanical composition is equal to the amounts in this range of Ficus rel. 2X no matter the source.

In another variation of the present embodiment, the botanical composition further comprises parts or extracts of Collinsonia Canadensis L. (“Collinsonia”). Collinsonia Canadensis L. is also known as Stone root or Canada Horsebalm. Typically, an extract of the root of Collinsonia Canadensis L is used herein. In a refinement, the extract is provided as a tincture, and in particular, a tincture in 65% alcohol/water (v/v). (v/v means volume/volume). Prior to forming the botanical composition of the invention, the tincture is optionally diluted with a suitable solvent (e.g., alcohol, water/alcohol). In one refinement, Collinsonia Canadensis L is present in an amount greater than or equal to, in increasing order of preference, 0.5 weight %, 0.005 weight %, 0.01 weight %, 0.03 weight %, 0.04 weight %, of the total weight of the botanical composition. In another refinement, Collinsonia Canadensis L is present in an amount less than or equal to, in increasing order of preference, 20 weight %, 10 weight %, 8 weight %, 5 weight %, and 2 weight % of the total weight of the botanical composition. The homeopathic composition Collinsonia 2X may be used as the source for Collinsonia Canadensis L. Therefore, in a variation of the present embodiment, the botanic composition includes Collinsonia 2X in an amount from about 1 to about 15 weight % of the total weight botanical composition. In a refinement of this variation, the amount of material from Collinsonia Canadensis L. in the botanical composition are equal to the amounts in this range of Collinsonia 2X no matter the source. In another variation of the present embodiment, the botanic composition includes Collinsonia 2X in an amount from about 4 to about 10 weight % of the total weight botanical composition. In a refinement of this variation, the amount of material from Collinsonia Canadensis L. in the botanical composition is equal to the amounts in this range of Collinsonia 2X no matter the source. In still another variation of the present embodiment, the botanic composition includes Collinsonia 2X in an amount from about 7 to about 9 weight % of the total weight botanical composition. In a refinement of this variation, the amount of material from Collinsonia Canadensis L. in the botanical composition is equal to the amounts in this range of Collinsonia 2X no matter the source.

In another variation of the present embodiment, the botanical composition further comprises parts or extracts of Aloe Socotrina. Aloe Socotrina, also known as Aloe vera, is a species of Aloe that is native to northern Africa. In a refinement, the extract is provided as a tincture, and in particular, a tincture in 65% alcohol/water (v/v). (v/v means volume/volume). Typically, the tincture made from the inspissated juice of Aloe Socotrina in 65% alcohol/water (v/v). Prior to forming the botanical composition of the invention, the tincture is optionally diluted with a suitable solvent (e.g., alcohol, water/alcohol). In one refinement, Aloe Socotrina is present in an amount greater than or equal to, in increasing order of preference, 0.1 weight %, 0.00005 weight %, 0.0001 weight %, 0.0003 weight %, 0.0004 weight %, of the total weight of the botanical composition. In another refinement, Aloe Socotrina is present in an amount less than or equal to, in increasing order of preference, 20 weight %, 10 weight %, 8 weight %, 5 weight %, and 2 weight % of the total weight of the botanical composition. The homeopathic composition Aloes Soc. 200 may be used as the source for Aloe Socotrina. Therefore, in a variation of the present embodiment, the botanic composition includes Aloes Soc. 200 in an amount from about 0.1 to about 10 weight % of the total weight botanical composition. In a refinement of this variation, the amount of material from Aloe Socotrina in the botanical composition are equal to the amounts in this range of Aloes Soc. 200 no matter the source. In another variation of the present embodiment, the botanic composition includes Aloes Soc. 200 in an amount from about 1 to about 8 weight % of the total weight botanical composition. In a refinement of this variation, the amount of material from Aloe Socotrina in the botanical composition is equal to the amounts in this range of Aloes Soc. 200 no matter the source. In still another variation of the present embodiment, the botanic composition includes Aloes Soc. 200 in an amount from about 2 to about 7 weight % of the total weight botanical composition. In a refinement of this variation, the amount of material from Aloe Socotrina in the botanical composition is equal to the amounts in this range of Aloes Soc. 200 no matter the source.

In another variation of the present embodiment, the botanical composition further comprises parts or extracts of Aesculus Hippocastanum L. Typically, an extract of the ripe nut (not including outside shell) Aesculus Hippocastanum L is used herein. Aesculus Hippocastanum L is also known as Horse Chestnut. In a refinement, the extract is provided as a tincture, and in particular, a tincture in 65% alcohol/water (v/v). (v/v means volume/volume). Prior to forming the botanical composition of the invention, the tincture is optionally diluted with a suitable solvent (e.g., alcohol, water/alcohol). In one refinement, Aesculus Hippocastanum L. is present in an amount greater than or equal to, in increasing order of preference, 0.1 weight %, 0.0001 weight %, 0.0005 weight %, 0.0007 weight %, 0.001 weight %, of the total weight of the botanical composition. In another refinement, Aesculus Hippocastanum L. is present in an amount less than or equal to, in increasing order of preference, 20 weight %, 10 weight %, 8 weight %, 5 weight %, and 2 weight % of the total weight of the botanical composition. The homeopathic composition Aesculus HIP. 200 may be used as the source for Aesculus Hippocastanum L. Therefore, in a variation of the present embodiment, the botanic composition includes Aesculus HIP. 200 in an amount from about 1 to about 15 weight % of the total weight botanical composition. In a refinement of this variation, the amount of material from Aesculus Hippocastanum L. in the botanical composition are equal to the amounts in this range of Aesculus HIP. 200 no matter the source. In another variation of the present embodiment, the botanic composition includes Aesculus HIP. 200 in an amount from about 4 to about 10 weight % of the total weight botanical composition. In a refinement of this variation, the amount of material from Aesculus Hippocastanum L. in the botanical composition is equal to the amounts in this range of Aesculus HIP. 200 no matter the source. In still another variation of the present embodiment, the botanic composition includes Aesculus HIP. 200 in an amount from about 7 to about 9 weight % of the total weight botanical composition. In a refinement of this variation, the amount of material from Aesculus Hippocastanum L. in the botanical composition is equal to the amounts in this range of Aesculus HIP. 200 no matter the source.

In another variation of the present embodiment, the botanical composition further comprises parts or extracts of Lapsana communis (“Lapsana com.). Lapsana communis is also known as Nipplewort or Dock-cress. In a refinement, the extract is provided as a tincture, and in particular, a tincture in 65% alcohol/water (v/v). (v/v means volume/volume). Prior to forming the botanical composition of the invention, the tincture is optionally diluted with a suitable solvent (e.g., alcohol, water/alcohol). In one refinement, Lapsana communis is present in an amount greater than or equal to, in increasing order of preference, 0.5 weight % 0.005 weight %, 0.01 weight %, 0.03 weight %, 0.04 weight % of the total weight of the botanical composition. In another refinement, Lapsana communis is present in an amount less than or equal to, in increasing order of preference, 20 weight %, 10 weight %, 8 weight %, 5 weight %, and 2 weight % of the total weight of the botanical composition. The homeopathic composition lapsana Com. 2X may be used as the source for Lapsana communis. Therefore, in a variation of the present embodiment, the botanic composition includes lapsana Com. 2X in an amount from about 0.1 to about 10 weight % of the total weight botanical composition. In a refinement of this variation, the amount of material from Lapsana communis in the botanical composition are equal to the amounts in this range of lapsana Com. 2X no matter the source. In another variation of the present embodiment, the botanic composition includes lapsana Com. 2X in an amount from about 1 to about 8 weight % of the total weight botanical composition. In a refinement of this variation, the amount of material from Lapsana communis in the botanical composition is equal to the amounts in this range of lapsana Com. 2X no matter the source. In still another variation of the present embodiment, the botanic composition includes lapsana Com. 2X in an amount from about 2 to about 7 weight % of the total weight botanical composition. In a refinement of this variation, the amount of material from Lapsana communis in the botanical composition is equal to the amounts in this range of lapsana Com. 2X no matter the source.

In another variation of the present embodiment, the botanical composition further comprises parts or extracts of Paeonia officinalis. Paeonia officinalis is also known as the common peony. Typically, an extract of the root (dug in spring) of Paeonia officinalis is used herein. In a refinement, the extract is provided as a tincture, and in particular, a tincture in 65% alcohol/water (v/v). (v/v means volume/volume). Prior to forming the botanical composition of the invention, the tincture is optionally diluted with a suitable solvent (e.g., alcohol, water/alcohol). In one refinement, Paeonia officinalis is present in an amount greater than or equal to, in increasing order of preference, 0.1 weight %, 0.00005 weight %, 0.0001 weight %, 0.0003 weight %, 0.0004 weight % the total weight of the botanical composition. In another refinement, Paeonia officinalis is present in an amount less than or equal to, in increasing order of preference, 20 weight %, 10 weight %, 8 weight %, 5 weight %, and 2 weight % of the total weight of the botanical composition. The homeopathic composition Paeonia Off. 30 may be used as the source for Paeonia officinalis. Therefore, in a variation of the present embodiment, the botanic composition includes Paeonia Off. 30 in an amount from about 0.1 to about 10 weight % of the total weight botanical composition. In a refinement of this variation, the amount of material from Paeonia officinalis in the botanical composition are equal to the amounts in this range of Paeonia Off. 30 no matter the source. In another variation of the present embodiment, the botanic composition includes Paeonia Off. 30 in an amount from about 1 to about 8 weight % of the total weight botanical composition. In a refinement of this variation, the amount of material from Paeonia officinalis in the botanical composition is equal to the amounts in this range of Paeonia Off. 30 no matter the source. In still another variation of the present embodiment, the botanic composition includes Paeonia Off. 30 in an amount from about 2 to about 7 weight % of the total weight botanical composition. In a refinement of this variation, the amount of material from Paeonia officinalis in the botanical composition is equal to the amounts in this range of Paeonia Off. 30 no matter the source.

In another variation of the present embodiment, the botanical composition further comprises parts or extracts of Achillea millefolium L. Achillea millefolium L is also known as Milfoil or Yarrow. Typically, an extract of the whole plant Achillea millefolium L is used herein. In a refinement, the extract is provided as a tincture, and in particular, a tincture in 65% alcohol/water (v/v). (v/v means volume/volume). Prior to forming the botanical composition of the invention, the tincture is optionally diluted with a suitable solvent (e.g., alcohol, water/alcohol). In one refinement, of Achillea millefolium L. is present in an amount greater than or equal to, in increasing order of preference, 0.1 weight %, 0.00005 weight %, 0.0001 weight %, 0.0003 weight %, 0.0004 weight % of the total weight of the botanical composition. In another refinement, of Achillea millefolium L. is present in an amount less than or equal to, in increasing order of preference, 20 weight %, 10 weight %, 8 weight %, 5 weight %, and 2 weight % of the total weight of the botanical composition. The homeopathic composition Millefolium 30 may be used as the source for Achillea millefolium L. Therefore, in a variation of the present embodiment, the botanic composition includes Millefolium 30 in an amount from about 0.1 to about 10 weight % of the total weight botanical composition. In a refinement of this variation, the amount of material from Achillea millefolium L. in the botanical composition are equal to the amounts in this range of Millefolium 30 no matter the source. In another variation of the present embodiment, the botanic composition includes Millefolium 30 in an amount from about 1 to about 8 weight % of the total weight botanical composition. In a refinement of this variation, the amount of material from Achillea millefolium L. in the botanical composition is equal to the amounts in this range of Millefolium 30 no matter the source. In still another variation of the present embodiment, the botanic composition includes Millefolium 30 in an amount from about 2 to about 7 weight % of the total weight botanical composition. In a refinement of this variation, the amount of material from Achillea millefolium L. in the botanical composition is equal to the amounts in this range of Millefolium 30 no matter the source.

In another variation of the present embodiment, the botanical composition further comprises parts or extracts of Strychnos nux-vomica L. Strychnos nux-vomica L. is also known as Poison nut or Quaker buttons. Typically, an extract of the seed (coarsely powdered) of Strychnos nux-vomica L. is used herein. In a refinement, the extract is provided as a tincture, and in particular, a tincture in 65% alcohol/water (v/v). (v/v means volume/volume). Prior to forming the botanical composition of the invention, the tincture is optionally diluted with a suitable solvent (e.g., alcohol, water/alcohol). In one refinement, Strychnos nux-vomica L. is present in an amount greater than or equal to, in increasing order of preference, 0.1 weight %, 0.00005 weight %, 0.0001 weight %, 0.0003 weight %, 0.0004 weight % of the total weight of the botanical composition. In another refinement, Strychnos nux-vomica L. is present in an amount less than or equal to, in increasing order of preference, 20 weight %, 10 weight %, 8 weight %, 5 weight %, and 2 weight % of the total weight of the botanical composition. The homeopathic composition Nux Vom. 200 may be used as the source for Strychnos nux-vomica L. Therefore, in a variation of the present embodiment, the botanic composition includes Nux Vom. 200 in an amount from about 0.1 to about 10 weight % of the total weight botanical composition. In a refinement of this variation, the amount of material from Strychnos nux-vomica L. in the botanical composition are equal to the amounts in this range of Nux Vom. 200 no matter the source. In another variation of the present embodiment, the botanic composition includes Nux Vom. 200 in an amount from about 1 to about 8 weight % of the total weight botanical composition. In a refinement of this variation, the amount of material from Strychnos nux-vomica L. in the botanical composition is equal to the amounts in this range of Nux Vom. 200 no matter the source. In still another variation of the present embodiment, the botanic composition includes Nux Vom. 200 in an amount from about 2 to about 7 weight % of the total weight botanical composition. In a refinement of this variation, the amount of material from Strychnos nux-vomica L. in the botanical composition is equal to the amounts in this range of Nux Vom. 200 no matter the source.

In another variation of the present embodiment, the botanical composition further comprises parts or extracts of Hydrastis canadensis L. Hydrastis canadensis L is also known as Golden seal, Orange root, Yellow root, and Indian turmeric. Typically, an extract of the rhizome and roots of Hydrastis canadensis L. is used herein. In a refinement, the extract is provided as a tincture, and in particular, a tincture in 65% alcohol/water (v/v). (v/v means volume/volume). Prior to forming the botanical composition of the invention, the tincture is optionally diluted with a suitable solvent (e.g., alcohol, water/alcohol). In one refinement, Hydrastis canadensis L. is present in an amount greater than or equal to, in increasing order of preference, 0.1 weight %, 0.00005 weight %, 0.0001 weight %, 0.0003 weight %, 0.0004 weight % of the total weight of the botanical composition. In another refinement, Hydrastis canadensis L is present in an amount less than or equal to, in increasing order of preference, 20 weight %, 10 weight %, 8 weight %, 5 weight %, and 2 weight % of the total weight of the botanical composition. The homeopathic composition Hydrastis Can 200 may be used as the source for Hydrastis canadensis L Therefore, in a variation of the present embodiment, the botanic composition includes Hydrastis Can 200 in an amount from about 0.1 to about 10 weight % of the total weight botanical composition. In a refinement of this variation, the amount of material from Hydrastis canadensis L. in the botanical composition are equal to the amounts in this range of Hydrastis Can 200 no matter the source. In another variation of the present embodiment, the botanic composition includes Hydrastis Can 200 in an amount from about 1 to about 8 weight % of the total weight botanical composition. In a refinement of this variation, the amount of material from Hydrastis canadensis L. in the botanical composition is equal to the amounts in this range of Hydrastis Can 200 no matter the source. In still another variation of the present embodiment, the botanic composition includes Hydrastis Can 200 in an amount from about 2 to about 7 weight % of the total weight botanical composition. In a refinement of this variation, the amount of material from Hydrastis canadensis L. in the botanical composition is equal to the amounts in this range of Hydrastis Can 200 no matter the source.

In another variation of the present embodiment, the botanical composition further comprises parts or extracts of Chelidonium majus L. Chelidonium majus L is also known as Greater Celandine. Typically, an extract of the whole plant of Chelidonium majus L. is used herein. In a refinement, the extract is provided as a tincture, and in particular, a tincture in 45% alcohol/water (v/v). (v/v means volume/volume). Prior to forming the botanical composition of the invention, the tincture is optionally diluted with a suitable solvent (e.g., alcohol, water/alcohol). In one refinement, Chelidonium majus L. is present in an amount greater than or equal to, in increasing order of preference, 0.1 weight %, 0.00005 weight %, 0.0001 weight %, 0.0003 weight %, 0.0004 weight %, of the total weight of the botanical composition. In another refinement, Chelidonium majus L. is present in an amount less than or equal to, in increasing order of preference, 20 weight %, 10 weight %, 8 weight %, 5 weight %, and 2 weight % of the total weight of the botanical composition. The homeopathic composition Chelidonium 1 M may be used as the source for Chelidonium majus L. Therefore, in a variation of the present embodiment, the botanic composition includes Chelidonium 1 M in an amount from about 0.1 to about 10 weight % of the total weight botanical composition. In a refinement of this variation, the amount of material from Chelidonium majus L. in the botanical composition are equal to the amounts in this range of Chelidonium 1 M no matter the source. In another variation of the present embodiment, the botanic composition includes Chelidonium 1 M in an amount from about 1 to about 8 weight % of the total weight botanical composition. In a refinement of this variation, the amount of material from Chelidonium majus L. in the botanical composition is equal to the amounts in this range of Chelidonium 1 M no matter the source. In still another variation of the present embodiment, the botanic composition includes Chelidonium 1 M in an amount from about 2 to about 7 weight % of the total weight botanical composition. In a refinement of this variation, the amount of material from Chelidonium majus L. in the botanical composition is equal to the amounts in this range of Chelidonium 1 M no matter the source.

In still another variation of the present invention, the botanical composition further comprises a component selected from the group consisting of nitric acid, potassium chloride, potassium phosphate, phosphate, ferrous phosphate, calcium fluoride, and combinations thereof. Potassium chloride may be provided in the form Kali muriaticum (“Kali Mur.”). Kali Mur. refers to a 10 wt % solution of potassium chloride (MW 74.55) in distilled water. In one refinement, Kali muriaticum is present in an amount greater than or equal to, in increasing order of preference, 0.1 weight %, 0.0001 weight %, 0.00001 weight %, 0.000001 weight %, 0.000005 weight % of the total weight of the botanical composition. In another refinement, Kali muriaticum is present in an amount less than or equal to, in increasing order of preference, 20 weight %, 10 weight %, 8 weight %, 5 weight %, and 2 weight % of the total weight of the botanical composition. Potassium chloride may be provided as Kali Mur. 6X. Therefore, in a variation of the present embodiment, the botanic composition includes Kali Mur. 6X in an amount from about 0.1 to about 10 weight % of the total weight botanical composition. In a refinement of this variation, the amount of potassium chloride. in the botanical composition are equal to the amounts in this range of Kali Mur. 6X no matter the source. In another variation of the present embodiment, the botanic composition includes Kali Mur. 6X in an amount from about 1 to about 8 weight % of the total weight botanical composition. In a refinement of this variation, the amount of potassium chloride in the botanical composition is equal to the amounts in this range of Kali Mur. 6X no matter the source. In still another variation of the present embodiment, the botanic composition includes Kali Mur. 6X in an amount from about 2 to about 7 weight % of the total weight botanical composition. In a refinement of this variation, the amount of potassium chloride in the botanical composition is equal to the amounts in this range of Kali Mur. 6X no matter the source.

Potassium phosphate may be provided in the form Kali phosphoricum (“Kali phos.”). Kali phos. is a 10 wt % solution of potassium phosphate (MW 174.18), dibasic in distilled water. In one refinement, Kali phosphoricum is present in an amount greater than or equal to, in increasing order of preference, 0.1 weight %, 0.0001 weight %, 0.00001 weight %, 0.000001 weight %, 0.000005 weight % of the total weight of the botanical composition. In another refinement, Kali phosphoricum is present in an amount less than or equal to, in increasing order of preference, 20 weight %, 10 weight %, 8 weight %, 5 weight %, and 2 weight % of the total weight of the botanical composition. Potassium phosphate may be provided as Kali phos. 6X. Therefore, in a variation of the present embodiment, the botanic composition includes Kali phos. 6X in an amount from about 0.1 to about 10 weight % of the total weight botanical composition. In a refinement of this variation, the amount of potassium phosphate in the botanical composition are equal to the amounts in this range of Kali phos. 6X no matter the source. In another variation of the present embodiment, the botanic composition includes Kali phos. 6X in an amount from about 1 to about 8 weight % of the total weight botanical composition. In a refinement of this variation, the amount of potassium phosphate in the botanical composition is equal to the amounts in this range of Kali phos. 6X no matter the source. In still another variation of the present embodiment, the botanic composition includes Kali phos. 6X in an amount from about 2 to about 7 weight % of the total weight botanical composition. In a refinement of this variation, the amount of potassium phosphate in the botanical composition is equal to the amounts in this range of Kali phos. 6X no matter the source.

Ferrous phosphate may be provided in the form of Ferrum phosphoricum (“Ferrum phos.”). Ferrous phosphate is also known as iron (II) phosphate. In one refinement, Ferrum phosphoricum is present in an amount greater than or equal to, in increasing order of preference, 0.1 weight %, 0.0001 weight %, 0.00001 weight %, 0.000001 weight %, 0.000005 weight % of the total weight of the botanical composition. In another refinement, Ferrum phosphoricum is present in an amount less than or equal to, in increasing order of preference, 20 weight %, 10 weight %, 8 weight %, 5 weight %, and 2 weight % of the total weight of the botanical composition. Ferrous phosphate may be provided as Ferrum phos. 6X. Therefore, in a variation of the present embodiment, the botanic composition includes Ferrum phos. 6X in an amount from about 0.1 to about 10 weight % of the total weight botanical composition. In a refinement of this variation, the amount of ferrous phosphate. in the botanical composition are equal to the amounts in this range of Ferrum phos. 6X no matter the source. In another variation of the present embodiment, the botanic composition includes Ferrum phos. 6X in an amount from about 1 to about 8 weight % of the total weight botanical composition. In a refinement of this variation, the amount of ferrous phosphate in the botanical composition is equal to the amounts in this range of Ferrum phos. 6X no matter the source. In still another variation of the present embodiment, the botanic composition includes Ferrum phos. 6X in an amount from about 2 to about 7 weight % of the total weight botanical composition. In a refinement of this variation, the amount of ferrous phosphate in the botanical composition is equal to the amounts in this range of Ferrum phos. 6X no matter the source.

Calcium fluoride may be provided in the form of Calcarea fluorica (“Calc. Flour.”). In one refinement, Calcarea fluorica is present in an amount greater than or equal to, in increasing order of preference, 0.1 weight %, 0.0001 weight %, 0.00001 weight %, 0.000001 weight %, 0.000005 weight % of the total weight of the botanical composition. In another refinement, Calcarea fluorica is present in an amount less than or equal to, in increasing order of preference, 20 weight %, 10 weight %, 8 weight %, 5 weight %, and 2 weight % of the total weight of the botanical composition. Calcium fluoride may be provided as Calcium Flour 6X. Therefore, in a variation of the present embodiment, the botanic composition includes Calcium Flour 6X in an amount from about 0.1 to about 10 weight % of the total weight botanical composition. In a refinement of this variation, the amount of calcium fluoride in the botanical composition are equal to the amounts in this range of Calcium Flour 6X no matter the source. In another variation of the present embodiment, the botanic composition includes Calcium Flour 6X in an amount from about 1 to about 8 weight % of the total weight botanical composition. In a refinement of this variation, the amount of calcium fluoride in the botanical composition is equal to the amounts in this range of Calcium Flour 6X no matter the source. In still another variation of the present embodiment, the botanic composition includes Calcium Flour 6X in an amount from about 2 to about 7 weight % of the total weight botanical composition. In a refinement of this variation, the amount of calcium fluoride in the botanical composition is equal to the amounts in this range of Calcium Flour 6X no matter the source.

Nitric acid may be provided in the form of Acid Nit 200. In one refinement, Acid Nit 200 is present in an amount greater than or equal to, in increasing order of preference, 0.1 weight %, 0.0001 weight %, 0.00001 weight %, 0.000001 weight %, 0.000005 weight % of the total weight of the botanical composition. In another refinement, Acid Nit 200 is present in an amount less than or equal to, in increasing order of preference, 20 weight %, 10 weight %, 8 weight %, 5 weight %, and 2 weight % of the total weight of the botanical composition. Nitric acid may be provided as Acid Nitric 200. Therefore, in a variation of the present embodiment, the botanic composition includes Acid Nitric 200 in an amount from about 0.1 to about 10 weight % of the total weight botanical composition. In a refinement of this variation, the amount of potassium phosphate in the botanical composition are equal to the amounts in this range of Acid Nitric 200 no matter the source. In another variation of the present embodiment, the botanic composition includes Acid Nitric 200 in an amount from about 1 to about 8 weight % of the total weight botanical composition. In a refinement of this variation, the amount of potassium phosphate in the botanical composition is equal to the amounts in this range of Acid Nitric 200 no matter the source. In still another variation of the present embodiment, the botanic composition includes Acid Nitric 200 in an amount from about 2 to about 7 weight % of the total weight botanical composition. In a refinement of this variation, the amount of potassium phosphate in the botanical composition is equal to the amounts in this range of Acid Nitric 200 no matter the source.

In yet another variation, the botanical composition is combined with one or more one or more pharmaceutically acceptable carriers or excipients. Suitable carriers and excipients are known to those skilled in the art of botanical and homeopathic compositions. Examples of such compositions include, but are not limited to, talc, sugar mannitol, lactose, pectin, dextrin, gelatin, starch, magnesium stearate, magnesium carbonate, sodium saccharine, cellulose, magnesium carbonate, sodium carboxymethylcellulose, a low melting wax, cocoa methylcellulose, tragacanth, butter, and the like. Naturally occurring forms of these materials are most desirable. In one refinement, the botanical composition includes an excipient or carrier in an amount of about 5 weight % to about 80 weight %.

In still another variation of the present invention, the botanical formulation is provided in an oral solid dosage form. In refinements of this variation, the botanical composition is provided as a solution, a suspension, a tablets, a pill, a capsule, a sustained release formulation, or a powder.

In another embodiment of the present invention, a method of alleviating one or more symptoms of hemorrhoids in a subject (i.e., a human patient) is provided. The method of this embodiment comprises administering to the subject a therapeutically effective amount of the botanical compositions set forth above. In one refinement, an amount from about 0.5 grains to about 12 grains of the botanical composition is administered to the subject per day. In another refinement, an amount from about 1 grains to about 8 grains of the botanical composition is administered to the subject per day. In another refinement, an amount from about 2 grains to about 6 grains of the botanical composition is administered to the subject each day. The composition will be administered to a subject for a sufficient number of day to alleviate a symptom of hemorrhoids. Typically, this time period is from about 3 days to about 6 months. In another refinement, the time period is from about 5 days to about 3 months. In still another refinement, the time period is from about 7 days to about 1 month.

The following examples illustrate the various embodiments of the present invention. Those skilled in the art will recognize many variations that are within the spirit of the present invention and scope of the claims.

### 1. Preparation

Mix together Negumdium 2× ( 5 ml), Ratanhia 200(5 ml), Acid Nit 200 (10 ml), Hamamelis 200(10 ml, Blumia OD 2×(10 ml, fiscus Rel. 2X(10 ml), Collinsonia 2X(10 ml), Aloes Soc. 200(5 ml), Aesculus HIP, 200(10 ml), Lapsana Com2X(5 ml), Paconia off.30(5 ml), Millifolium 30(5 ml), Nux Vom 200 (5 ml), Hydrastic Can 200 (5ml), Chelidonium 1M and Mix Kali phos 6, calc. Flour. 6, Kali Mur. 6, and Ferrum phos, in equal quantity with above all mixture with Plumbum met 3× with sufficient absorption of trituration to prepare a two grain tablet.

2. Study Relief of Pain and Bleeding Associated with Hemorrhoids

The purpose of the study was to investigate the effects of the botanical combination for the relief of pain and bleeding associated with hemorrhoids. The nutritional supplement described herein as NBR/OAPI/01/02 contained 15 botanical extracts, including Aesculus hippocastanum, and was administered as tablets to 40 adult hemorrhoid patients whose chief symptoms were pain and bleeding. The composition of NBR/OAPI/01/02 is shown below in Table 1.

TABLE 1
Composition of NBR/OAPI/01/02 for 1000 - 65 mg tablets

| Active Ingredient | Amount (ml) |
|---|---|
| Negundium (2x) | 5 |
| Ratanhia (200) | 5 |
| Acid Nitric (200) | 10 |
| Hamamelis (200) | 10 |
| Blumia OD (2x) | 10 |
| Ficus Rel. (2x) | 10 |
| Collinsonia (2x) | 10 |
| Aloes. Soc. (200) | 5 |
| Aesculus Hippocastanum (200) | 10 |
| Lapsana Com (2x) | 5 |
| Paeonia Off. (30) | 5 |
| Millifolium (30) | 5 |
| Nux. Vom. (200) | 5 |
| Hydrastis Can (200) | 5 |
| Chelidonium (1 M) | 5 |
| Kali phosphate (6x) | 5 |
| Calcium Flour (6x) | 5 |
| Kali Mur. (6x) | 5 |
| Ferrum phosphate (6x) | 5 |

### Methods

Thirteen investigators participated in the open label, multi-center study of NBR/OAPI/01/02, a botanical treatment compound. They included patients who were diagnosed with hemorrhoids (piles), including patients with prolapse, fissure, fistula, rectal polyps, etc. All 40 subjects were treated with four tablets every three hours. The endpoints measured were “Pain Relieved” and “Bleeding Relieved” on a scale of 0-100% improvement. The patients were instructed to report any adverse events and complications.

### Results

The average individual improvement of 33 patients taking NBR/OAPI/01/02 was 55.38%. The total number of patients responding to treatment (31 pts) of those patients who met study qualifications (33 pts) was calculated to produce an efficacy result of 93.9%. A total of 35 patients presented with bleeding and 30 (86%) found relief with NBR/OAPI/01/02. A total of 30 patients presented with pain and 25 (83%) found relief with NBR/OAPI/01/02. A summary of individual symptoms and improvements are shown below in Table 2.

TABLE 2
Efficacy Results for Individual Symptoms

| | | Total No. | | | |
|---|---|---|---|---|---|
| | | of Pts w/ | | No | |
| No. | Symptom | symptom | Aggravation | Change | Amelioration |
| 1 | Bleeding | 35 | 3 | 2 | 30 |
| 2 | Pain | 30 | 3 | 2 | 25 |
| 3 | Swelling | 9 | 0 | 7 | 2 |
| 4 | First Degree | 36 | 4 | 7 | 25 |
| | Piles* | | | | |
| 5 | Second Degree | 14 | 0 | 7 | 7 |
| | Piles** | | | | |
| 6 | Third Degree | 3 | 0 | 1 | 2 |
| | Piles*** | | | | |
| 7 | Anal fistula, | 3 | 0 | 2 | 1 |
| | anal fissure, | | | | |
| | etc. | | | | |
| 8 | Other | 0 | 0 | 0 | 0 |
| | Complications | | | | |

*Piles with bleeding
**Piles with prolapse but retract spontaneously
***Piles require manual placement after prolapsing

The botanical combination present in NBR/OAPI/01/02 is effective in relieving pain and bleeding associated with hemorrhoids. NBR/OAPI/01/02 is definitely active as shown by an improvement of patients with hemorrhoids by 55.38%. However, the treatment was not found to be effective for prolapse cases, among the patients included in this investigation, the percentage of patients with prolapse was 26%.

This study concluded that NBR/OAPI/01/02 is quite effective for the relief of pain and bleeding associated with hemorrhoids (piles). The preliminary overall effectiveness of NBR/OAPI/01/02 was determined in the absence of a placebo group. Additional controlled studies are indicated including chronic patients with complications such as prolapse, fissure, fistula and rectal polyps.

While embodiments of the invention have been illustrated and described, it is not intended that these embodiments illustrate and describe all possible forms of the invention. Rather, the words used in the specification are words of description rather than limitation, and it is understood that various changes may be made without departing from the spirit and scope of the invention.

What is claimed is:

1. A botanical composition comprising:
a therapeutically effective amount of an admixture comprising:
an extract from the bark of Negundo aceroides; and
parts or extracts of Blumea odorata.

2. The botanical composition of claim 1 further comprising parts or extracts of the root of Krameria triandra Ruiz.

3. The botanical composition of claim 1 further comprising parts or extracts of the bark of Hamamelis virginica.

4. The botanical composition of claim 1 further comprising parts or extracts of Ficus religiosa L.

5. The botanical composition of claim 1 further comprising parts or extracts of Collinsonia Canadensis L.

6. The botanical composition of claim 1 further comprising parts or extracts of Aloe Socotrina.

7. The botanical composition of claim 1 further comprising parts or extracts of Aesculus Hippocastanum L.

8. The botanical composition of claim 1 further comprising parts or extracts Lapsana communis.

9. The botanical composition of claim 1 further comprising parts or extracts of Paeonia officinalis.

10. The botanical composition of claim 1 further comprising parts or extracts of Achillea millefoliumn L.

11. The botanical composition of claim 1 further comprising parts or extracts of Strychnos nux-vomica L.

12. The botanical composition of claim 1 further comprising parts or extracts of Hydrastis canadensis L.

13. The botanical composition of claim 1 further comprising parts or extracts of Chelidonium majus L.

14. The botanical composition of claim 1 further comprising a component selected from the group consisting of nitric acid, potassium chloride, potassium phosphate, phosphate, calcium fluoride, and combinations thereof.

15. A botanical composition comprising:
a therapeutically effective amount of:
an extract from the bark of Negundo aceroides; and
parts or extracts of Blumea odorata;
parts or extracts of the root of Krameria triandra Ruiz;
parts or extracts of the bark of Hamamelis virginica;
parts or extracts of Ficus religiosa L.;
parts or extracts of Collinsonia Canadensis L.;
parts or extracts of Aloe Socotrina.
parts or extracts of Aesculus Hippocastanum L.;
parts or extracts Lapsana communis; and
parts or extracts of Paeonia officinalis; and
one or more pharmaceutically acceptable carriers or excipients.

16. The botanical composition of claim 1 further comprising parts or extracts of Achillea millefolium L

17. The botanical composition of claim 1 further comprising parts or extracts of Strychnos nux-vomica L.

18. The botanical composition of claim 1 further comprising parts or extracts of Hydrastis canadensis L.

19. The botanical composition of claim 1 further comprising parts or extracts of Chelidonium majus L.

20. The botanical composition of claim 1 further comprising a component selected from the group consisting of nitric acid, potassium chloride, potassium phosphate, phosphate, calcium fluoride, and combinations thereof.

21. A method of alleviating a symptom of hemorrhoids in a subject, the method comprising:
administering to the subject a therapeutically effective amount of a botanical composition comprising:
an extract from the bark of Negundo aceroides; and
parts or extracts of Blumea odorata.
